(19) United States
(12) Patent Application Publication
(10) Pub. No.: US 2018/0147083 A1
(43) Pub. Date: May 31, 2018

(54) MEDICAL ENDOVAGINAL DEVICE

(71) Applicant: Martina MILANESI, Arezzo (IT)

(72) Inventor: Martina MILANESI, Arezzo (IT)

(21) Appl. No.: 15/580,743

(22) PCT Filed: Jun. 8, 2016
(86) PCT No.: PCT/IB2016/000772

§ 371 Date: Dec. 8, 2017

(30) Foreign Application Priority Data
Jun. 8, 2015 (IT) 102015000021094

(51) Int. Cl. A61F 5/41

(52) U.S. Cl. CPC A61F 5/41

(57) ABSTRACT

Disclosed is a medical endovaginal device, including an elongated element defining a direction of development and having, at its proximal end, a head arranged to be introduced inside the vagina and, at its distal end, engagement element with the extreme portion of the penis during sexual intercourse.

The present invention relates to a medical endovaginal device.

As well as being a physical impairment, an inadequate penis size is often a source of psychological distress that can undermine relations between the partners and causes great social discomfort.

It is well known that the morphology and function of the male external genital organ affect, throughout life, the state of perception of health or psychophysical well-being, the sex and couple life, as well as, more generally, the relational life. It therefore plays a key role at behavioral level in sexual and interpersonal relationships.

According to recent literature, the average length of the erect penis is about 13 cm. In medicine, there are congenital or acquired diseases (so-called idiopathic micropenis, partial amputation of the penis, etc.), which have well-defined clinical pictures of micropenis or microphallus, with the penis in erection lower than the abovementioned average.

Another increasingly common problem concerns the sex change (transgender) and subsequent reconstructive surgery of male genitals (neophallus) through phalloplasty interventions that can lead to the creation of penises of reduced size.

To date, usually it is done by surgery.

One of the most widely used is the penile suspensory ligament section that allows a certain penile lengthening in a “flaccidity” condition, which consists basically in cutting the suspensory ligament of the penis from the pubic symphysis, allowing a forward movement of the corpora cavernosa.

In addition to the surgical trauma to the patient, such intervention can lead to possible impairment of stability penile during erection and subsequent loss of the so-called angle of erection.

Moreover, among other possible complications there is also the risk of a reattachment of this ligament by a reattachment of the sectioned margin of the ligament itself, resulting in a shortening of the penis itself (relapse).

Another surgical lengthening method, often performed along with the penile suspensory ligament section, comprises a skin plastic surgery carried out on the advancement flaps of the peno-pubic skin on the penis.

Possible complications are especially the loss of the distal flap, poor wound healing, dehiscence of the wound itself, as well as the production of pathological scarring with fibrotic retraction.

On the other hand, as regards to the extension, in the state of the art there are numerous methods of incremental phalloplasty, for example comprising the use of penile prostheses, each suffering from different percentages of success and complications.

One of the safest and effective methods to increase penile size and increase the girth of the penis is the “lipofilling” method by using fat (autologous fat tissue previously aspirated and properly treated) as filling material injected into the treatment area.

However, one of the limitations of this technique, which among other things is very expensive, is the fact that large corrections are not possible, and multiple interventions of this type are often needed.

Another problem related to phalloplasty is given by the fact that the lipofilling involves a circumferential increase of 20/30% which weighs down the penis itself resulting in an increase in the length of about 20-30% compared to penile size, which is why this method it should be associated with the section of the penile suspensory ligament.

However, both the surgical approach and the phalloplasty, in addition to involving the above concerns, are partial solutions that require corrections and controls.

In addition, there are cases where the penis is not so small as to require surgery, but it is inadequate because it has a shorter length in erection compared to the average population.

The aim of the invention is to eliminate the drawbacks described above in known types of medical endovaginal device enabling the disabled person to have a full sexual intercourse that is satisfactory for the person and the partner.

Within the above aim, an object of the present invention is to provide a medical endovaginal device, which does not require surgical interventions and which is also applicable to penises of inadequate length.

Another object of the invention is that it has low costs.

Another object of the present invention is to allow the couple satisfaction in a complete hygienic safety condition.

Another object of the invention is to provide a medical endovaginal device by using readily commercially available means and commonly used materials, so that it is economically competitive.

This aim, as well as these and other objects which will become apparent hereinafter, are achieved by a medical endovaginal device, according to the invention, characterized in that it comprises an elongated element defining a direction of development and having, at its proximal end, a head arranged to be introduced into the vagina and, at its distal end, engagement means with the extreme portion of the penis during sexual intercourse.

Further features and advantages of the invention will be more apparent from the description of a preferred but not exclusive embodiment of the medical endovaginal device, illustrated by way of non-limiting example with the aid of the accompanying drawings, wherein:

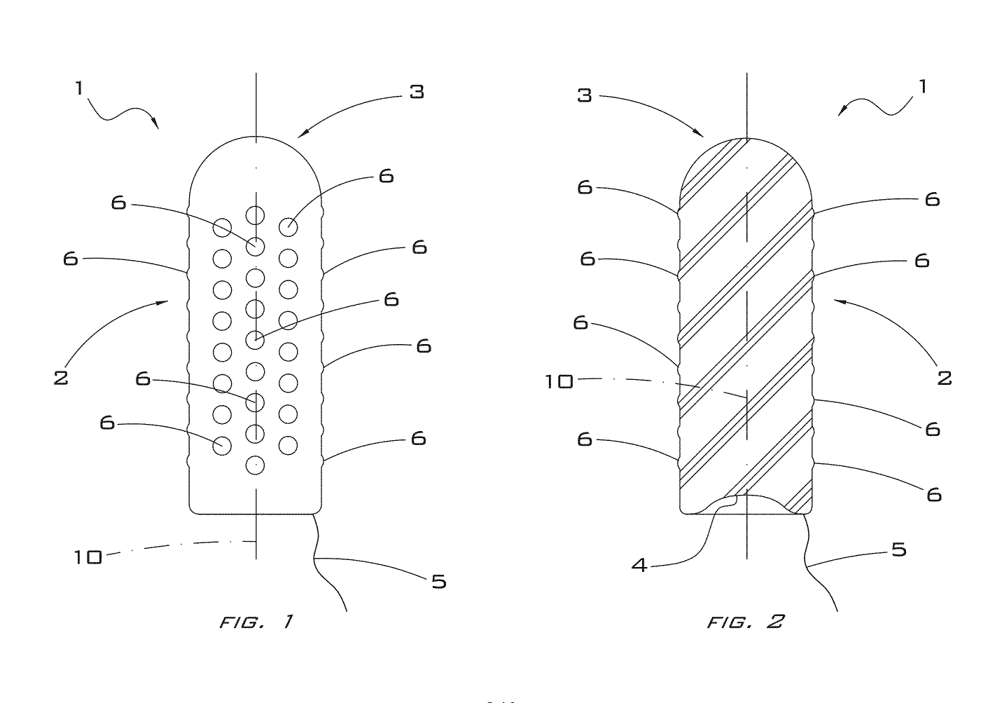

FIG. 1 shows a first variant of the medical endovaginal device (1), in elevation;

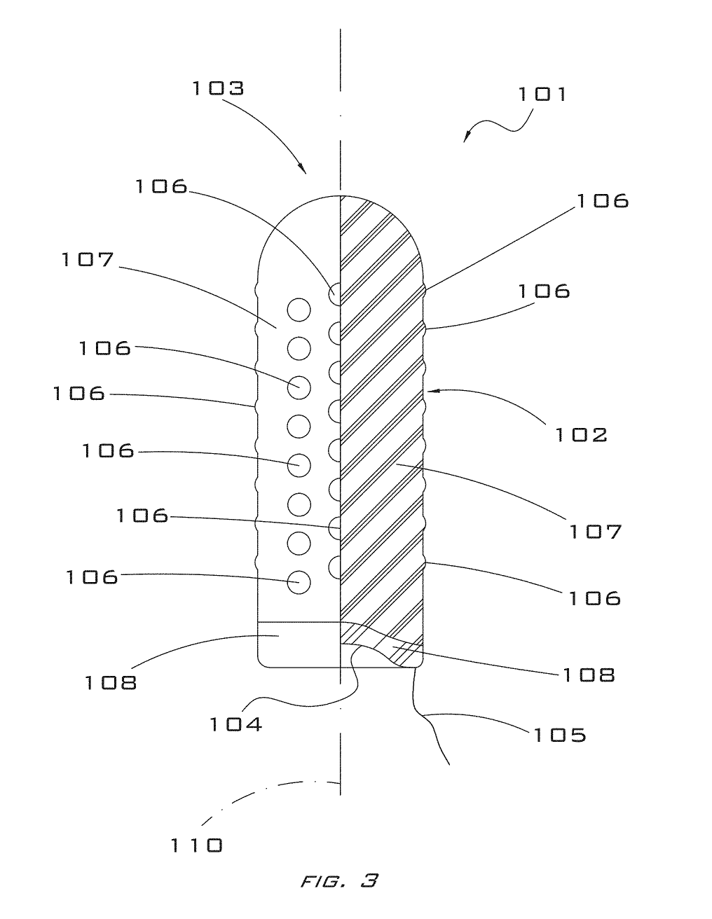

FIG. 2 shows the device (1) of FIG. 1, sectioned with a vertical plane relative to the head (3) of the device (1), passing through its direction of development (10);

FIG. 3 shows a second variant of the device (101), represented in elevation and sectioned vertically with respect to the head (103);

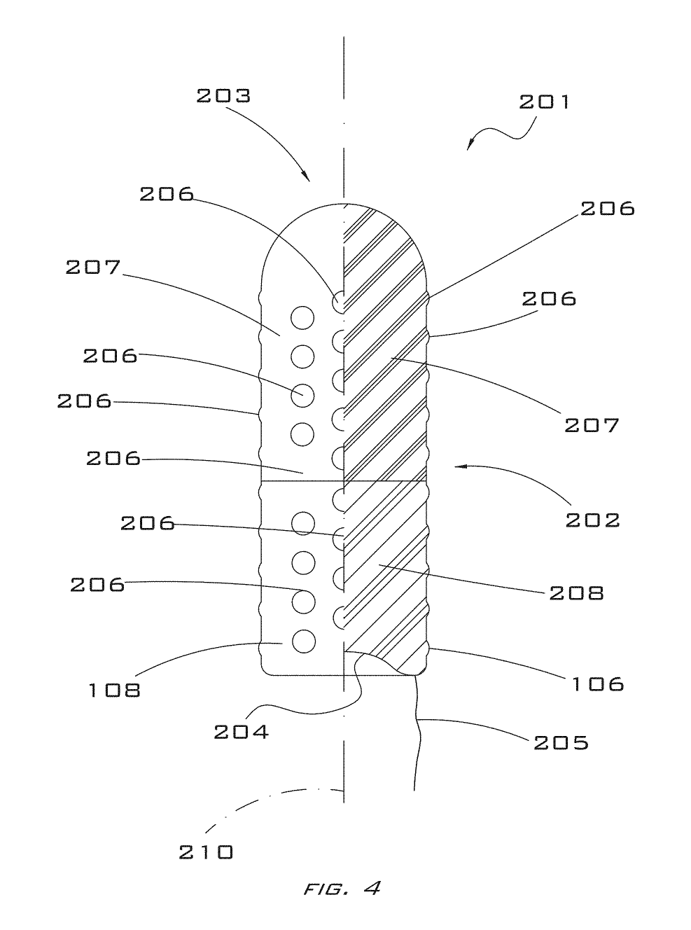

FIG. 4 is a third embodiment of the device (201), represented in elevation and sectioned vertically with respect to the head (203);

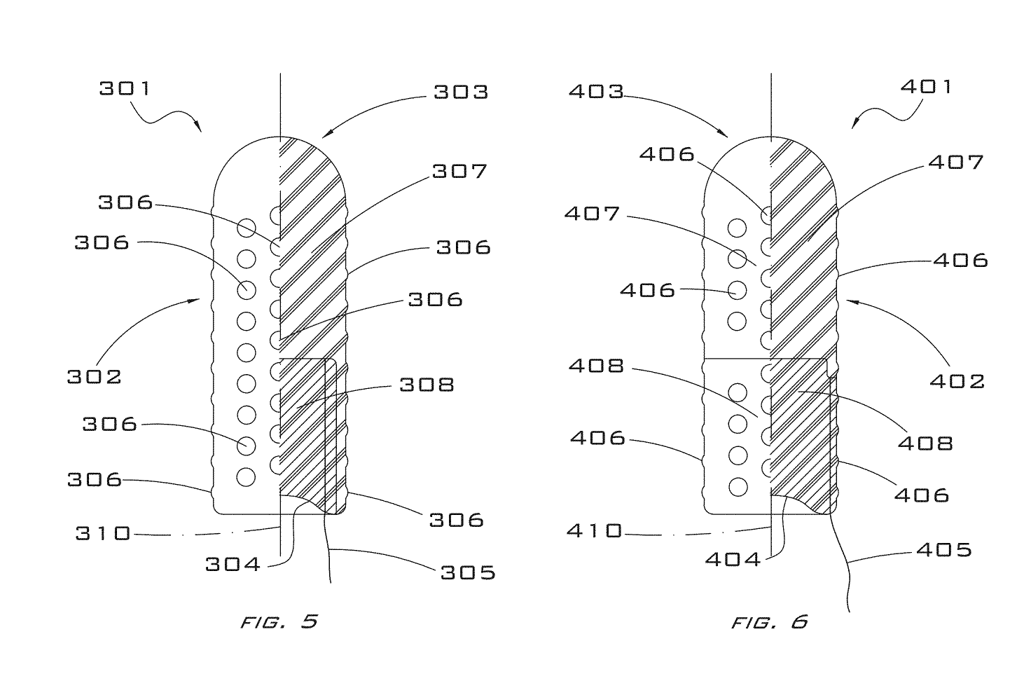

FIG. 5 represents a fourth example of the device (301), represented in elevation and sectioned vertically with respect to the head (303);

FIG. 6 represents a last variation of the device (401), represented in elevation and sectioned vertically with respect to the head (403).

With reference to the above figures, a medical endovaginal device according to the invention, which is generally referred to by the numerals 1, 101, 201, 301, 401, has the basic features of comprising a substantially cylindrical elongated element 2, 102, 202, 302, 402, defining a direction of development 10, 110, 210, 310, 410 and having, at its proximal end, a head 3, 103, 203, 303, 403 arranged to be introduced inside the vagina and, at its distal end, engagement means 4, 104, 204, 304, 404 with the extreme portion of the penis during sexual intercourse.

The head 3, 103, 203, 303 and 403, for example, has a hemispherical shape.

Advantageously, said engagement means comprise a hollow support 4, 104, 204, 304 and 404 for the penis during sexual intercourse, so that the thrust towards the inside of the vagina is more effective and which does not give problems to the penis itself or discomfort to its owner.

Necessarily, the elongate element 2, 102, 202, 302 and 402 is made of a biocompatible material, for example silicone or silicone gel.

Depending on the material chosen, the device can be washable and reusable after disinfection with antibacterial solutions and detergents, or it can be disposable.

Advantageously, the elongate element 2, 102, 202, 302 and 402 has different mechanical characteristics along the direction of development 10, 110, 210, 310 and 410, in such a way that it is stiffer towards its proximal end and less stiff (semi-rigid) towards its distal end.

In this way, the device 1, 101, 201, 301 and 401 will be suitable to stimulate more effectively the uterine neck of the vagina due to the extreme stiffness of the head 3, 103, 203, 303 and 403, but it will not be uncomfortable to the partner of the woman pushing the extreme part of his penis with the hollow portion 4, 104, 204, 304 and 404.

With reference to FIG. 3, the device 101 includes a thickness 108 for supporting the extreme portion of the penis during sexual intercourse, connected to the distal end of said elongated body 102.

The thickness 108 is defined the hollow portion 104 for engaging the penis.

Preferably, the elongated element 102 is made of silicone, while the thickness 108 of made of silicone gel.

In other embodiments 201, 301 and 401, the elongated element 202, 302 and 402 includes a proximal portion 207, 307 and 407, and a distal portion 208, 308 and 408, solidly associated with each other, for example by gluing or melting. The two portions 207, 307, 407th 208, 308 and 408 have different stiffnesses, in a way that the device 201, 301 and 401 has a greater rigidity toward the head 203, 303 and 403, and a lower rigidity at the hollow portion 204, 304 and 404, respectively defined on the outer face of the distal portions 208, 308 and 408.

An embodiment provides that the proximal portions 207, 307 and 407 are made of silicone, while the distal portions 208, 308 and 408 are made of silicone gel, or however of a biocompatible material with tactile characteristics similar to those of the vaginal bottom.

In a variant embodiment, the proximal and distal portions, 207 and 208, are glued to each other along a flat face (FIG. 4), in a second variant the distal portion 308 is inserted into the proximal portion 308 (FIG. 5), and in still another variant the two portions 407 and 408 are glued on two distinct planes (FIG. 6).

The latter configuration allows an easier positioning of the recovery means 405 of the device 406.

Advantageously, indeed, recovery means 5, 105, 205, 305 and 405 of the device 1, 101, 201, 301 and 401 are provided, such as a thread 5, 105, 205, 305, 405 associated to the elongated element 2, 102, 202, 302, 402.

For security reasons, each of the threads 205, 305 and 405 are to be associated to the relative proximal portion 207, 307 and 407. For the same reason, also the thread 105 has been preferably associated to the elongated element 102 rather than to the thickness 108.

In the embodiments 201 and 401 (FIGS. 4 and 6), since the outer surface which faces towards the male penis consists only and exclusively in the external face of the corresponding portion 208 and 408, a communication channel between the interface between the portions 207 and 208, 407 and 408 can be provided, within their respective distal portions 208 and 408.

Advantageously, the side surface of the device 1, 101, 201, 301 and 401 has a set of protuberances 6, 106, 206, 306 and 406 for the stimulation of the vaginal wall by contact.

Preferably, vaginal lubricant releasing means may be provided, so that the device 1, 101, 201, 301 and 401 can be used to improve the lubrication of the vagina, especially in women suffering from vaginal dryness.

Spermicide releasing means can also provided, so that the device 1, 101, 201, 301 and 401 can also be used as an effective contraceptive.

On the contrary, at least one seminal fluid conveying channel can be provided, which is defined along the direction of development of the elongated element, to facilitate the fertilization of the woman.

An external applicator can be also provided, which is similar to that employed in the introduction of tampons, for the correct insertion of the inventive device.

In practice, the device 1, 101, 201, 301 and 401 is to be introduced into the vagina prior to the sexual intercourse, also by means of the external applicator, until the head 3, 103, 203, 303 and 403 reaches the bottom (uterine neck).

Once the sexual intercourse is started, with the penis penetrating the vagina and engaging the hollow portion 4, 104, 204, 304 and 404, the device 1, 101, 201, 301 and 401 acts as an extension, and even if the penis is small, it can give pleasure to the woman.

Obviously, depending on the length of the penis or on the length of the uterine neck, the size of the device 1, 101, 201, 301 and 401 itself varies accordingly.

To extract the device 1, 101, 201, 301 and 401, it is only required to pull the thread 5, 105, 205, 305 and 405 outward.

From the above description it is thus clear that the invention achieves the intended aim and objects, and in particular the fact is stressed that a medical endovaginal device is provided, which allows the disabled person, with reduced dimensions of the penis, to have a full sexual intercourse that is satisfactory for the person and the partner.

In particular, the idea of a device to be introduced into the vagina, thus shifting the solution of the problem on the woman, allows to avoid all the above mentioned surgical procedure problems as well as numerous female sexual disorders, such as sexual arousal disorders and the orgasmic disorder, which can be overcome in a particular way by conforming the outer surface of the elongated element, for example with the use of the above set of projections, but also to treat or alleviate the sexual pain disorders, in particular vaginismus or dyspareunia, by “training” the vaginal conduit to hold the device in its interior.

Moreover, the vaginal lubricant releasing means ameliorate all the disorders and discomfort due to the lack of lubrication.

In addition, the biocompatible material used for its construction allows a safe use in terms of risk of infections or allergies.

Another advantage of the medical endovaginal device is given by the fact that, acting as a mechanical plug for the whole duration of the sexual intercourse, allows a safe and clean penetration even during the menstrual cycle of the woman, without the risk that leaks can contact the male penis.

Another advantage of the invention is given by the fact that such a device has low production costs, and so it will be also possible to realize it in disposable form by suitably choosing the material, with the maximum guarantees from a hygiene point of view.

Moreover, the idea of a softer side to supporting the penis and a more rigid side to going against the uterine neck ensures a comfortable use ensuring the pleasure of the woman.

Another advantage of the invention is that, thanks to the set of protuberances defined on the outer surface of the elongated element, it can be used as recreational and entertainment tool by all couples, even without particular and specific male disabilities to be overcome.

Another advantage of the device according to the invention is given by the fact that it can be used as a valid means of contraception, or, by changing its conformation, as a fertilization aid.

Last but not least, the use of readily commercially available means and the use of common materials make the device economically competitive. The so-designed invention is susceptible of numerous modifications and variations, all of which fall within the scope of the inventive concept. Further, all the details will be replaced by other technically equivalent elements.

In practice, the materials employed, as well as the dimensions, may be different according to requirements, provided they are consistent with the realization purpose.

1. A medical endovaginal device (1, 101, 201, 301, 401), comprising an elongated element (2, 102, 202, 302, 402) defining a direction of development (10, 110, 210, 310, 410) and having, at its proximal end, a head (3, 103, 203, 303, 403) arranged to be introduced inside the vagina and, at its distal end, engagement means (4, 104, 204, 304, 404) with the extreme portion of the penis during sexual intercourse.

2. Device according to claim 1, wherein said engagement means (4, 104, 204, 304, 404) comprise a hollow support (4, 104, 204, 304, 404) for the penis.

3. Device according to claim 1, wherein said elongated element (2, 102, 202, 302, 402) is made of silicone gel.

4. Device according to claim 1, wherein said elongated element (2, 102, 202, 302, 402) has different mechanical characteristics along said development direction (10, 110, 210, 310, 410), in such a way that it is more rigid towards its proximal end and less rigid toward its distal end.

5. Device according to claim 1, further comprising a thickness (108) for supporting the end portion of the penis during sexual intercourse, connected to the distal end of said elongated body (102).

6. Device according to claim 1, wherein said elongated element (202, 302, 402) comprises a proximal portion (207, 307, 407) and a distal portion (208, 308, 408) solidly associated with each other and having different stiffness, in a way that the device (201, 301, 401) has a greater rigidity in proximity of said head (203, 303, 403) than the stiffness at its distal end.

7. Device according to claim 1, further comprising recovery means (5, 105, 205, 305, 405) of the device (1, 101, 201, 301, 401).

8. Device according to claim 1, wherein said recovery means (5, 105, 205, 305, 405) comprise a thread (5, 105, 205, 305, 405) associated with said elongated element (2, 102, 202, 302, 402).

9. Device according to claim 6, wherein said thread (5, 105, 205, 305, 405) is associated to said proximal portion (207, 307, 407).

10. Device according to claim 1, further comprising means for release of a spermicide substance, in such a way that the device (1, 101, 201, 301, 401) is a means of contraception.

11. Device according to claim 2, wherein said elongated element (2, 102, 202, 302, 402) is made of silicone gel.

12. Device according to claim 2, wherein said elongated element (2, 102, 202, 302, 402) has different mechanical characteristics along said development direction (10, 110, 210, 310, 410), in such a way that it is more rigid towards its proximal end and less rigid toward its distal end.

13. Device according to claim 3, wherein said elongated element (2, 102, 202, 302, 402) has different mechanical characteristics along said development direction (10, 110, 210, 310, 410), in such a way that it is more rigid towards its proximal end and less rigid toward its distal end.

14. Device according to claim 2, further comprising a thickness (108) for supporting the end portion of the penis during sexual intercourse, connected to the distal end of said elongated body (102).

15. Device according to claim 3, further comprising a thickness (108) for supporting the end portion of the penis during sexual intercourse, connected to the distal end of said elongated body (102).

16. Device according to claim 4, further comprising a thickness (108) for supporting the end portion of the penis during sexual intercourse, connected to the distal end of said elongated body (102).

17. Device according to claim 2, wherein said elongated element (202, 302, 402) comprises a proximal portion (207, 307, 407) and a distal portion (208, 308, 408) solidly associated with each other and having different stiffness, in a way that the device (201, 301, 401) has a greater rigidity in proximity of said head (203, 303, 403) than the stiffness at its distal end.

18. Device according to claim 3, wherein said elongated element (202, 302, 402) comprises a proximal portion (207, 307, 407) and a distal portion (208, 308, 408) solidly associated with each other and having different stiffness, in a way that the device (201, 301, 401) has a greater rigidity in proximity of said head (203, 303, 403) than the stiffness at its distal end.

19. Device according to claim 4, wherein said elongated element (202, 302, 402) comprises a proximal portion (207, 307, 407) and a distal portion (208, 308, 408) solidly associated with each other and having different stiffness, in a way that the device (201, 301, 401) has a greater rigidity in proximity of said head (203, 303, 403) than the stiffness at its distal end.

20. Device according to claim 5, wherein said elongated element (202, 302, 402) comprises a proximal portion (207, 307, 407) and a distal portion (208, 308, 408) solidly associated with each other and having different stiffness, in a way that the device (201, 301, 401) has a greater rigidity in proximity of said head (203, 303, 403) than the stiffness at its distal end.
